(19) United States
(12) Patent Application Publication
(10) Pub. No.: US 2010/0104635 A1
(43) Pub. Date: Apr. 29, 2010

(54) PROCESS FOR PREPARING GRANULES OF HYDROPHILIC VITAMINS

(76) Inventors: Duane Hoagland, Wilmington, NC (US); Anisul Quadir, Hackettstown, NJ (US); Charles Onyiuke, Mt. Arlington, NJ (US); Frank Tranor, Wilmington, NJ (US); Yoshitomi Kakiguchi, Hyogo (JP); Tomoyoshi Kajiura, Tokyo (JP); Kai Zhuang, River Edge, NJ (US)

Correspondence Address: CONNOLLY BOVE LODGE & HUTZ, LLP, P O BOX 2207, WILMINGTON, DE, 19899, US

(21) Appl. No.: 12/441,212

(22) PCT Filed: Sep. 14, 2007
(86) PCT No.: PCT/EP2007/059705

§ 371 Date: Dec. 22, 2009

Related U.S. Application Data
(60) Provisional application No. 60/845,249, filed on Sep. 15, 2006.
(60) Provisional application No. 60/862,314, filed on Oct. 20, 2006.
(60) Provisional application No. 60/865,715, filed on Nov. 14, 2006.
(60) Provisional application No. 60/951,837, filed on Jul. 25, 2007.

(51) Int. Cl. A61K 9/32; A61K 31/341

(52) U.S. Cl. 424/464; 424/497; 514/474

(57) ABSTRACT

The present invention relates to a process for preparing granules of a hydrophilic vitamin with polyvinylpyrrolidone as binder in a fluidized bed granulator, to the granules of a hydrophilic vitamin obtained by said process, to tablets made with said granules of a hydrophilic vitamin, to mixture of L-ascorbic acid for direct tabletting and tablets made with said mixtures.

The present invention relates to a process for preparing granules of a hydrophilic vitamin with polyvinylpyrrolidone as binder in a fluidized bed granulator, to the granules of a hydrophilic vitamin obtained by said process and to tablets made with said granules of a hydrophilic vitamin.

In order to create a usable form of a hydrophilic vitamin like L-ascorbic acid (vitamin C) that can be efficiently and successfully tabletted for commercial human consumption, the hydrophilic vitamin crystals obtained by synthesis or from natural sources have to be granulated. In a common granulation process for vitamins the hydrophilic vitamin powder is granulated with a solution of a binder in a granulator or in a fluidized bed.

U.S. Pat. No. 4,036,948 describes the granulation of L-ascorbic acid in a fluidized bed granulation apparatus wherein the L-ascorbic acid powder is spray-coated with a solution of a binder. In the examples different starches, hydroxypropylmethylcellulose and ethylcellulose have been used as binders. It is described that the binder is used in an amount of 2 to 4 weight percent relative to L-ascorbic acid.

DE 3447423 A 1 describes a process for the production of ascorbic acid granules using polyvinylpyrrolidone as binder in a fluidized bed granulator. Preferred is the use of a mixture of a soluble polyvinylpyrrolidone and an insoluble polyvinylpyrrolidone. The lowest total amount of polyvinylpyrrolidone used in the examples is slightly above 2 weight percent.

It is an object of the present invention to provide a process for preparing granules of a hydrophilic vitamin, preferably L-ascorbic acid granules having a higher content of water soluble vitamin, in particular L-ascorbic acid, compared to commercial products, that means higher than 98 weight percent, while maintaining the properties of the presently used commercial granules with respect to flowability, homogeneity, color stability, tablet hardness and tablet strength. It is another object of the present invention to provide granules of a hydrophilic vitamin, in particular L-ascorbic acid granules having said properties. The increased amount of hydrophilic vitamin in the granules allows the formulator to reduce the size of the tablet while maintaining the amount of granules of a hydrophilic vitamin or to add additional ingredients to a multi vitamin tablet.

We have found that this object is achieved by a process for preparing granules of a hydrophilic vitamin for direct compression which comprises spray-coating powder of a hydrophilic vitamin, having a size such that not lower than 80 weight percent of the powder passes through a 200-mesh screen (75 μm screen), with a solution of polyvinylpyrrolidone having a K-value from 70 to 100 under constant agitation in a fluidized bed granulator until the total amount of polyvinylpyrrolidone has reached about 0.5 to 1.9 weight percent relative to the sum of polyvinylpyrrolidone and hydrophilic vitamin and wherein the amount of hydrophilic vitamin in the final granules is at least 98.1 weight percent.

A person skilled in the art is familiar with the term direct compression. Direct compression or direct tabletting means the compression of a powder mixture without previous granulation of the powder mixture. It is known that many hydrophilic vitamins, particularly L-ascorbic acid, have to be transformed to direct compressible vitamin granules as described in the present invention or in the above mentioned documents (U.S. Pat. No. 4,036,948 and DE 3447423 A 1).

In the present invention the term hydrophilic vitamin stands for thiamin (B1), riboflavin (B2), niotinamide, pantothenic acid, pyridoxine (B6), cobalamin (B12), folic acid, L-ascorbic acid (C) or biotin, preferably thiamin (B1), pyridoxine (B6), cobalamin (B12) or L-ascorbic acid, most preferably L-ascorbic acid. The term L-ascorbic acid stands not only for the acid itself but also for its ascorbates, particularly its alkali metal salts or its alkaline earth metal salts. Preferably L-ascorbic acid stands for the acid itself, its sodium salt or its calcium salt, most preferably for the acid itself.

In the present invention, use is made of a powder of a hydrophilic vitamin, in particular L-ascorbic acid powder, as starting material, of which not less than 80 weight percent preferably not less than 85 weight percent passes through a 200-mesh screen (75 μm screen).

A person skilled in the art knows the fluid bed granulation process, which is also known as agglomeration. This process involves suspending particulates in an air stream and spraying a liquid from the top down onto the fluidized bed. Particles in the path of the spray get slightly wetted and become tacky. The tacky particles collide with other particles and adhere to them to form a granule. The solvent is evaporated in the warm stream of air.

In the process of the present invention the liquid, which is sprayed onto the fluidized bed of L-ascorbic acid powder is a solution of polyvinylpyrrolidone. The polyvinylpyrrolidone has a K-value from 70 to 100, preferably from 80 to 98, most preferably from 90 to 98, in particular from 93 to 96. The polyvinylpyrrolidone of the spray solution is a soluble polymer, in particular a soluble homopolymer.

The K-value is calculated from the measured relative viscosity of a 1% solution of polyvinylpyrrolidone in water according to literature (H. Fikentscher, Cellulosechemie 13 (1932) 58-64 and 71-74; monographs of the Ph.Eur and USP Pharmocopoeisas on Povidone).

A K-value range of 80 to 98 of a polyvinylpyrrolidone homopolymer corresponds to a weight average of the molecular weight of about 1 000 000 to 1 500 000 g/mol (V. Bühler, “Polyvinylpyrrolidone—Excipients for Pharmaceuticals”, Springer-Verlag Berlin Heidelberg 2005; page 23 to page 25).

In principle polyvinylpyrrolidone is soluble in many different solvents, which can be evaporated under the conditions of the process, like ethanol, isopropanol, chloroform, acetic acid or water. In the process of the present invention water is the preferred solvent for polyvinylpyrrolidinone in order to form the spray solution.

The solution of polyvinylpyrrolidone used in the process of the present invention is preferably an aqueous solution of polyvinylpyrrolidinone homopolymer.

The solution of polyvinylpyrrolidone, preferably the aqueous solution of polyvinylpyrrolidone can be stabilized with a preservative in order to avoid the growth of microorganisms like fungi, yeast or bacteria during storage. Examples of preservatives are parabens, sorbic acid and its salts or benzoic acid and its salts.

The preferred preservative for the aqueous solution of polyvinylpyrrolidone is sorbic acid or its salts. In case of sorbic acid and its salts, like calcium sorbate or potassium sorbate, the pH-value of the aqueous solution of polyvinylpyrrolidone is usually below 6.5, preferably below 5.

The aqueous solution of polyvinylpyrrolidone used in the inventive process can be obtaineded directly from the polymerization of vinylpyrrolidone or it can be obtained by dissolving a dry powder of the appropriate polyvinylpyrrolidone in water, preferably directly before using the solution in the fluidized bed granulation process.

The concentration of polyvinylpyrrolidone in the solution for spray coating is usually in the range of 1 to 20 weight percent, preferably from 2 to 10 weight percent, most preferably from 3 to 7 weight percent.

The granulation process continues until the total amount of polyvinylpyrrolidone has reached about 0.5 to 1.9 weight percent relative to the sum of polyvinylpyrrolidone and the hydrophilic vitamin. The total amount of polyvinylpyrrolidone in the final product preferably ranges from 0.8 to 1.6, particularly from 0.9 to 1.2, weight percent relative to the sum of polyvinylpyrrolidone and hydrophilic vitamin.

The total polyvinylpyrrolidone of the final granules obtained by the process of the present invention preferably originates predominantly from the spray solution. That means that preferably at least 80, in particularly at least 90, most preferably at least 95 weight percent of the total polyvinylpyrrolidone of the final granules are soluble.

The amount of the hydrophilic vitamin, in particularly of L-ascorbic acid in the final granules is at least 98.1 weight percent, in particularly at least 98.4 weight percent, most preferably at least 98.8 weight percent.

The granules formed in the fluid bed granulation process are usually dried for a while after the complete addition of the spray solution in order to obtain particles with a defined solvent residue.

The final granules of the process of the present invention preferably have been dried up to a solvent-content of less than 1 weight percent, in particularly up to less than 0.7 weight percent, most preferably up to less than 0.4 weight percent. The solvent content of the final granules was determined by drying the granules for 2 hours at 105° C. under static conditions.

Another object of the present invention are granules of a hydrophilic vitamin produced by spray-coating powder of a hydrophilic vitamin, having a size such that not lower than 80 weight percent of the powder passes through a 200-mesh screen (75 μm screen), with a solution of polyvinylpyrrolidone having a K-value from 70 to 100 under constant agitation in a fluidized bed granulator until the total amount of polyvinylpyrrolidone has reached about 0.5 to 1.9 weight percent relative to the sum of polyvinylpyrrolidone and hydrophilic vitamin and wherein the amount of hydrophilic vitamin in the final granules is at least 98.1 weight percent.

The granules of a hydrophilic vitamin are particularly L-ascorbic acid granules.

The preferred features and ranges of the granules of a hydrophilic vitamin, in particular of L-ascorbic acid granules, with respect to polyvinylpyrrolidinone, the K-value of the polyvinylpyrrolidone, the amount of the polyvinylpyrrolidone, the solvent of the spray solution, the amount of solvent residues and the amount of hydrophilic vitamin, in particular L-ascorbic acid, are defined as described above.

Another object of the present invention are vitamin tablets produced by direct compression of the above-described granules of a hydrophilic vitamin, in particular L-ascorbic acid granules, which have been produced by the process of the present invention.

The inventive granules of a hydrophilic vitamin, in particular L-ascorbic acid granules, are mixed with common auxiliaries and eventually with additional active ingredients like other direct compressible vitamin powders in order to form a mixture, which is used in a process of direct tabletting.

Common vitamin C tablets comprise 500 or 1000 mg L-ascorbic acid. The formulation of a 500 mg vitamin C tablet usually differs from the formulation of a 1000 mg vitamin C tablet.

The inventive granules of a hydrophilic vitamin, in particular L-ascorbic acid granules are also useful for direct tabletting of 1000 mg vitamin tablets, in particular 1000 mg vitamin C tablets.

Common auxiliaries for direct tabletting are described in DE 3447423 A 1, in U.S. Pat. No. 4,036,948 and in the book titled “Vademecum for vitamin formulations” by Volker Bühler, Wiss. Verl.-Ges. Stuttgart, 1988. Usually the hydrophilic vitamin mixture for direct tabletting comprises besides the granulate of the hydrophilic vitamin at least one, preferably more than two further excipients.

Tablets, which are made from mixtures of L-ascorbic acid for direct tabletting have to show good properties with respect to tablet hardness, tablet friability and tablet disintegration time. It is also an important feature, that the mixtures of L-ascorbic acid for direct tabletting can be used on modern high-speed tabletting machines.

Another aspect of the present invention relates to mixtures of L-ascorbic acid for direct tabletting.

A mixture for direct tabletting consisting only of L-ascorbic acid granules for direct compression, preferably the inventive L-ascorbic acid granules, comprising at least 90%, preferably at least 94%, more preferably at least 96%, in particular at least 98.5% by weight of L-ascorbic acid, from 0.25 to 0.75%, preferably from 0.4 to 0.6% by weight of the final mixture of stearic acid, from 6 to 20%, preferably from 10 to 14% or from 2 to 20%, preferably from 3 to 15%, more preferably from 4 to 12% by weight of the final mixture of a microcrystalline cellulose, wherein the average particle size of the microcrystalline cellulose is usually in the range from 5 to 500 μm, preferably from 10 to 250 μm, in particular from 30 to 100 μm, like Avicel® PH 101, and optionally at least one disintegrant selected from the group of disintegrants consisting of low-substituted hydroxypropylcellulose, carboxymethylcellulose, croscarmelose, carmelose, crospovidone and sodium starch glycolate, preferably croscarmelose, crospovidone and sodium starch glycolate, can be easily compressed to tablets that show good properties with respect to hardness and friability. No capping was observed.

Also preferred is a mixture for direct tabletting consisting of L-ascorbic acid granules for direct compression, from 2 to 16%, preferably 4 to 12% by weight of the final mixture of a microcrystalline cellulose, wherein the average particle size of the microcrystalline cellulose is usually in the range from 5 to 500 μm, preferably from 10 to 250 μm, in particular from 30 to 100 μm, like Avicel® PH 101, at least one lubricant, preferably selected from the group of lubricants consisting of stearic acid and its derivatives, for example magnesium stearate or calcium stearate, and talc, a polyethylene glycol, e.g. PEG 6000, a poloxamer, e.g. poloxamer 188, calcium arachinate and sodium stearyl fumerate, more preferably stearic acid and magnesium stearate, in particular stearic acid, wherein the lubricant is used preferably in an amount from 0.2 to 2%, more preferably from 0.3 to 0.8%, in particular from 0.4 to 0.6% by weight of the final mixture, and optionally at least one disintegrant selected from the group of disintegrants consisting of low-substituted hydroxypropylcellulose, carboxymethylcellulose, croscarmelose, carmelose, crospovidone and sodium starch glycolate, preferably croscarmelose, crospovidone and sodium starch glycolate, wherein the content of L-ascorbic acid is between 82 to 95%, preferably 85 to 94%, in particular 88 to 93% by weight of the final mixture.

The L-ascorbic acid granules for direct compression that can be used in the two above-discussed mixtures for direct tabletting are for example the ascorbic acid granules described in U.S. Pat. No. 4,036,948 or DE 3447423 A 1.

The L-ascorbic acid granules for direct compression can be prepared for example by a fluidized granulation process as described in the present application, by a compaction process like roller compaction or by using a mechanical mixer like a ploughshare mixer. The L-ascorbic acid granules usually comprise as a water-soluble binder for example pregelatinized starch (e.g. corn starch or potato starch), pregelatinized modified starch, water-soluble cellulose (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum or gelatin and as organic solvent-soluble binders for example cellulose derivatives (e.g. cellulose acetate phthalate, hydroxypropylmethyl-cellulose phthalate, ethyl-cellulose).

In case a disintegrant is used in the two above-discussed mixtures for direct tabletting the disintegrant is preferably used in an amount from 0.1 to 5%, preferably from 0.5 to 4%, in particular from 1 to 3% by weight of the final mixture.

Another aspect of the present invention relates to L-ascorbic acid tablets, which are produced by direct compression and which consist essentially of one of the two above-mentioned mixtures for direct tabletting.

The present invention is described further in the following examples, which are presented solely for the non-limiting purpose of further illustrating the invention.

### EXAMPLES

### Example 1

99 parts of ascorbic acid, which were milled and passed to 85% through a 200 mesh screen (75 micron screen), were sprayed under constant agitation in a fluidized bed granulator with an aqueous 5% solution of polyvinylpyrrolidone (prepared from Luviskol® K90 solution approx. 20%) until the amount of polyvinylpyrrolidone had reached 1 part, followed by drying in situ. The conditions of the granulation process were:

| | |
|---|---|
| Inlet temperature: | 80° C. to 90° C. |
| Inlet air rate: | 3.5 to 6 m³/sec |
| Spraying time: | 55 to 75 min |
| Drying time: | 2 to 10 min |
| Final water content: | less than 0.15% (after 2 hours at 105° C.) |

The material was sifted in a rotary sifter. The over 20 mesh (850 μm) material was removed and comminuted in a knife mill. The 20 mesh through material and the milled oversize were combined to form the product stream.

Physical Data of the Granules:

| | | |
|---|---|---|
| Actual concentration of binder | 1.06 | weight percent |
| (polyvinylpyrrolidon) | | |
| Bulk density | 0.74 | g/ml |
| Tap density | 0.77 | g/ml |

Particle Size Analysis:

| | | |
|---|---|---|
| 20 mesh | 850 μm | 0 |
| 35 mesh | 500 μm | 17.2% |
| 40 mesh | 425 μm | 11.9% |
| 60 mesh | 250 μm | 27.6% |
| 80 mesh | 180 μm | 16.1% |
| 100 mesh | 150 μm | 6.7% |
| pan | less than 150 μm | 18.1% |
| Carr index (%) | 3.90 | |

### Example 2

99.2 parts of the granules prepared in example 1 were mixed with 0.8 parts of magnesium stearate. The mixture was compressed into tablets. The given values of the tablets represent the average values of 20 tablets.

| | | |
|---|---|---|
| Compression force: | 12 | kN |
| Average weight: | 489.3 | mg |
| Average thickness: | 4.73 | mm |
| Average hardness: | 4.7 | kp |
| Friability: | 1.9% | loss |
| Compression force: | 15 | kN |
| Average weight: | 502.6 | mg |
| Average thickness: | 4.71 | mm |
| Average hardness: | 5.0 | kp |
| Friability: | 1.1% | loss |

### Example 3

Using a process similar to example 1, 98.5 parts of ascorbic acid, which were milled and passed to 85% through a 200 mesh screen (75 micron screen), were sprayed under constant agitation in a fluidized bed granulator with an aqueous 5% solution of polyvinylpyrrolidone (prepared from Luviskol® K90 solution approx. 20%) until the amount of polyvinylpyrrolidone had reached 1.5 parts, followed by drying in situ. The conditions of the granulation process with exception of the spraying time, which had to be adjusted, were the same as in example 1:

The material was sifted to remove oversize (+20 mesh=>850 μm) and fines (−100 mesh=<150 μm)

Physical Data of the Granules:

| | |
|---|---|
| Bulk density | 0.58 g/ml |
| Tap density | 0.60 g/ml |

Particle Size Analysis:

| | | |
|---|---|---|
| 20 mesh | 850 μm | 0.49 |
| 40 mesh | 425 μm | 40.98% |
| 60 mesh | 250 μm | 39.02% |
| 80 mesh | 180 μm | 14.15% |
| 100 mesh | 150 μm | 3.90% |
| pan | less than 150 μm | 1.46% |

### Example 4

507.61 g of the granules prepared in example 3 were mixed with 4.09 g of magnesium stearate. The mixture was compressed into tablets. The given values of the tablets represent the average values of 10 tabletts.

| | | |
|---|---|---|
| Compression force: | 12 | kN |
| Average weight: | 527 | mg |
| Average thickness: | 5.0 | mm |
| Average hardness: | 5.89 | kp |
| Friability: | 1.0% | loss (20 tablets) |
| Compression force: | 15 | kN |
| Average weight: | 540 | mg |
| Average thickness: | 5.0 | mm |
| Average hardness: | 8.3 | kp |
| Friability: | 0.4% | loss (20 tablets) |

### Example 5

Using a process similar to Example 1, 97 parts of ascorbic acid, which were milled and passed to 85% through a 200 mesh screen (75 micron screen), were sprayed under constant agitation in a fluidized bed granulator with an aqueous 5 weight percent solution of corn starch (prepared from corn starch and water as described in example 1 of U.S. Pat. No. 4,036,948) until the amount of corn starch had reached 3 parts, followed by drying in situ. The conditions of the granulation process with exception of the spraying time, which had to be adjusted, were the same as in Example 1:

### Example 6

L-ascorbic acid granules were obtained by the same manner as described in Example 5, wherein 97 parts of ascorbic acid were treated with an aqueous solution of hydroxypropylmethyl-cellulose until the amount of hydroxypropylmethyl-cellulose had reached 3 parts.

### Example 7

87.68 parts of the granules prepared in example 5 were mixed with 11.82 parts of microcrystalline cellulose (mean particle size 50 μm, e.g. Avicel® PH 101) and 0.5 parts of stearic acid. The mixture was compressed into tablets. The given values of the tablets represent the average values of 10 tablets.

| Compression force: | 13 | kN |
|---|---|---|
| Average weight: | 606 | mg |
| Average thickness: | not determined | |
| Average hardness: | 9.4 | kp |
| Friability: | not determined | |
| Compression force: | 15 | kN |
| Average weight: | 603 | mg |
| Average thickness: | 5.5 | mm |
| Average hardness: | 10.6 | kp |
| Friability: | 0.4% | loss |

### Example 8

87.68 parts of the granules prepared in example 6 were mixed with 11.82 parts of microcrystalline cellulose (mean particle size 50 μm, e.g. Avicel® PH 101) and 0.5 parts of stearic acid. The mixture was compressed into tablets. The given values of the tablets represent the average values of 10 tablets.

| Compression force: | 13 | kN |
|---|---|---|
| Average weight: | 583 | mg |
| Average thickness: | not determined | |
| Average hardness: | 8.6 | kp |
| Friability: | not determined | |
| Compression force: | 15 | kN |
| Average weight: | 585 | mg |
| Average thickness: | not determined | |
| Average hardness: | 10.1 | kp |
| Friability: | 0.7% | loss |

### Example 9

87.68 parts of the granules prepared in example 1 were mixed with 11.82 parts of microcrystalline cellulose (mean particle size 50 μm, e.g. Avicel® PH 101) and 0.5 parts of stearic acid. The mixture was compressed into tablets. The given values of the tablets represent the average values of 20 tablets.

| Compression force: | 13 | kN |
|---|---|---|
| Average weight: | 589 | mg |
| Average thickness: | 5.3 | mm |
| Average hardness: | 8.8 | kp |
| Friability: | not determined | |
| Compression force: | 15 | kN |
| Average weight: | 578 | mg |
| Average thickness: | 5.2 | mm |
| Average hardness: | 9.5 | kp |
| Friability: | 1.0% | loss |

### Example 10

93.0 parts of the granules prepared in example 1 were mixed with 6.5 parts of microcrystalline cellulose (mean particle size 50 μm) and 0.5 parts of stearic acid. The mixture was compressed into tablets. The given values of the tablets represent the average values of 10 tablets.

| Compression force: | 13 | kN |
|---|---|---|
| Average weight: | 589 | mg |
| Average hardness: | 5.9 | kp |
| Friability: | not determined | |
| Compression force: | 15 | kN |
| Average weight: | 578 | mg |
| Average hardness: | 9.2 | kp |
| Friability: | 1.0% | loss |

All references described above are incorporated by references in their entirety for all useful purposes.

1-14. (canceled)

15. A process for preparing granules of a hydrophilic vitamin for direct compression comprising spray-coating powder of a hydrophilic vitamin, said powder having a size such that not lower than 80 weight percent of the powder passes through a 200-mesh screen (75 μm screen), with a solution of polyvinylpyrrolidone having a K-value of from 70 to 100 under constant agitation in a fluidized bed granulator until the total amount of polyvinylpyrrolidone has reached from about 0.5 to 1.9 weight percent relative to the total weight of polyvinylpyrrolidone and hydrophilic vitamin and wherein the amount of hydrophilic vitamin in the final granules is at least 98.1 weight percent.

16. The process of claim 15, wherein said hydrophilic vitamin is L-ascorbic acid.

17. The process of claim 15, wherein the K-value of the polyvinylpyrrolidone is from 90 to 98.

18. The process of claim 15, wherein the total amount of polyvinylpyrrolidone is from 0.8 to 1.6 weight percent relative to the total weight of polyvinylpyrrolidone and hydrophilic vitamin.

19. The process of claim 15, wherein the final granules have been dried up to a solvent-content of less than 1 weight percent.

20. A granule of a hydrophilic vitamin, wherein said granule is produced by spray-coating powder of a hydrophilic vitamin, said powder having a size such that not lower than 80 weight percent of the powder passes through a 200-mesh screen (75 μm screen), with a solution of polyvinylpyrrolidone having a K-value of from 70 to 100 under constant agitation in a fluidized bed granulator until the total amount of polyvinylpyrrolidone has reached from about 0.5 to 1.9 weight percent relative to the total weight of polyvinylpyrrolidone and hydrophilic vitamin and wherein the amount of hydrophilic vitamin in the final granule is at least 98.1 weight percent.

21. The granule of claim 20, wherein said hydrophilic vitamin is L-ascorbic acid.

22. The granule of claim 20, wherein the K-value of the polyvinylpyrrolidone is from 90 to 98.

23. The granule of claim 20, wherein the amount of polyvinylpyrrolidone is from 0.8 to 1.6 weight percent relative to the total weight of polyvinylpyrrolidone and hydrophilic vitamin.

24. The granule of claim 20, wherein said granule has a solvent-content of less than 1 weight percent.

25. A vitamin tablet produced by directly compressing one or more granules of claim 20.

26. A mixture for direct tabletting, said mixture consisting of (1) L-ascorbic acid granules for direct compression, wherein said granules comprise at least 90% by weight of L-ascorbic acid, (2) from 0.25 to 0.75% by weight, based on the total weight of said mixture, of stearic acid, (3) from 2 to 20% by weight, based on the total weight of said mixture, of a microcrystalline cellulose, and (4) optionally at least one disintegrant selected from the group consisting of tow-substituted hydroxypropyl cellulose, carboxymethylcellulose, croscarmelose, carmelose, crospovidone, and sodium starch glycolate.

27. A mixture for direct tabletting, said mixture consisting of (1) L-ascorbic acid granules for direct compression, (2) from 2 to 16% by weight, based on the total weight of said mixture, of a microcrystalline cellulose, (3) at least one lubricant, and (4) optionally at least one disintegrant selected from the group consisting of low-substituted hydroxypropylcellulose, carboxymethylcellulose, croscarmelose, carmelose, crospovidone, and sodium starch glycolate, and wherein the content of L-ascorbic acid in said mixture is from 82 to 95% by weight, based on the total weight of said mixture.

28. An L-ascorbic acid tablet produced by directly compressing a mixture, wherein said mixture consists essentially of the mixture of claim 26.
